(19) United States
(12) Patent Application Publication
(10) Pub. No.: US 2006/0193428 A1
(43) Pub. Date: Aug. 31, 2006

(54) Control arrangement for a computed tomography apparatus and method for controlling a computed tomography apparatus

(75) Inventor: Bjorn Heismann, Erlangen (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengesellschaft

(21) Appl. No.: 11/361,251

(22) Filed: Feb. 23, 2006

(30) Foreign Application Priority Data
Feb. 28, 2005 (DE) 10 2005 009 085.0

(51) Int. Cl. H05G 1/60; A61B 6/00; G01N 23/00; G21K 1/12

(52) U.S. Cl. 378/4

(57) ABSTRACT

In a control device for a computed tomography apparatus and method for controlling a computer tomography apparatus, the control device acquires a number of temporally successive volume data sets of the same subject in order to detect a temporal variation of the measurement subject. During the acquisition of a further volume data set of the same measurement subject, using the x-ray radiation currently received by the receiver unit, the control device automatically establishes a correlation in real time between a stored, earlier volume data set of the same measurement subject and the volume data set to be directly acquired, and the control device controls the x-ray source such that the x-ray radiation used for the acquisition of the volume data set to be directly acquired exhibits an intensity and/or dose that is inversely dependent on the established correlation.

### BACKGROUND OF THE INVENTION

1. Field of the Invention

The present invention concerns a control arrangement for a computed tomography apparatus of the type wherein the intensity and/or dose of the emitted x-ray radiation is/are controlled dependent on radiation detected by the imaging radiation detector, as well as a method for controlling a computed tomography apparatus.

2. Description of the Prior Art

Computed tomography apparatuses are used today for various medical examinations. X-ray radiation is used which has the capability to at least partially penetrate solid bodies, in particular non-metallic bodies. Upon passage of the radiation through an examination subject, the radiation is partially absorbed dependent on the structural composition of the subject, such that the intensity of the radiation after passage through the subject is lower than before the passage. This effect is designated as attenuation. After the penetration through the examination subject, the radiation can be detected, for example, by a scintillator detector, and the attenuation of the radiation can be determined by comparison of the measured radiation with the radiation emitted by an x-ray source. Insights about the distribution of material within the subject can be acquired in this manner.

The use of x-ray radiation (which is intrinsic to the system in computed tomography apparatuses), it is a disadvantage that x-rays can damage biological tissue at a certain dose. In medical computed tomography it is therefore desirable to keep the radiation dose required for a measurement low.

In medical technology, computed tomography systems typically are used in order to create volume exposures of the human body. The x-ray source and a reception device (detector) of the computed tomography apparatus are moved around the (optimally stationary) examination subject during a scan to acquire a volume dataset.

The quality of the images that can be generated by means of the volume dataset is decisive for the diagnostic value of a computed tomography apparatus. The use of x-ray radiation with high intensity generally leads to a better image quality but results in a higher x-ray dose exposure for a patient.

In known computed tomography systems, a value for the intensity of the radiation to be emitted by the x-ray source is established by a user before beginning a scan. It is known to not predetermine this value as fixed, but rather to allow for certain fluctuations during the rotation of x-ray source and reception device of the computed tomography apparatus around the subject. For example, various aspect ratios of the patient can be taken into account, such that the exposure of the patient can be reduced.

A computed tomography apparatus and a method for controlling a computed tomography apparatus are known from DE 102 25 188 A1. To control the computed tomography apparatus for acquisition of projections of a subject, it is proposed to initially predetermine a desired value for a factor characteristic for the quality of the projections to be acquired by the computed tomography apparatus, and subsequently to acquire sequential projections of the subject and thereby to determine a real value of the characteristic factor for the quality of the respective projection between the acquisition of successive projections. The primary intensity of an x-ray source of the computed tomography apparatus between the acquisition of successive projections is subsequently regulated such that the real value of the characteristic factor of the projections acquired by the computed tomography apparatus is held to the predetermined desired value. The x-ray dose exposure for the subject should be kept optimally low. In summary, this known method proposes to adjust in real time the intensity of the x-ray source of the computed tomography apparatus dependent on the individual projection data forming a volume dataset, such that a predetermined image quality is achieved.

A method and apparatus for automatic exposure control in computed tomography is known from the DE 103 44 357 A1. In this known computed tomography apparatus, an exposure control device calculates an actual attenuation profile of an x-ray slice through the patient during a first half of a rotation of the x-ray focal point around a patient, starting from electrical signals that are generated by a radiation detection device of the computed tomography apparatus during the first half of the rotation. Using these data, an extrapolated attenuation profile for a second half of the rotation is calculated in advance. This extrapolated attenuation profile calculated in advance is used for regulation of an operating parameter of the x-ray source in order to regulate the radiation dose that is emitted by the x-ray source during the second half of the rotation. In summary, this method thus discloses initially acquiring half of a volume dataset using a predetermined x-ray current for the x-ray source of the computed tomography apparatus, and to adapt this x-ray current using the information obtained in the first half of the volume dataset to acquire the second half of the volume dataset. This procedure is based on the recognition that, for a stationary subject, the x-ray radiation for the second half of the volume dataset irradiates a volume that exhibits a mirror-inverted structure relative to the volume irradiated during the acquisition of the first half of the volume dataset.

Although these known methods reduce the radiation exposure of the patient that occurs during the acquisition of a volume dataset, the radiation exposure for a patient being examined by computed tomography is generally still too high in order to medically justify the acquisition of multiple volume datasets of same subject in short intervals. The reason is that, in all known approaches, the total x-ray exposure upon acquisition of multiple volume datasets is a sum of constant individual x-ray exposures that respectively occur in the acquisition of a volume dataset.

Modern computed tomography apparatuses exhibit a slice coverage of 5 to 6 cm. This means that they can acquire data from a volume of 5 to 6 cm in thickness in one revolution around the subject, and therewith forming one volume dataset. The trend is moving toward ever-larger coverages, with a coverage of, for example, 12 cm already being realized. Given such a coverage, the acquisition of a complete organ (such as, for example, of the human heart), or at least of significant parts of human organs, is possible by means of one revolution of the x-ray source and the reception device of the computed tomography apparatus, and thus by means of a single volume dataset.

Due to the large x-ray radiation exposure discussed above, computed tomography apparatuses normally are only used for acquisition of static images.

In medicine, however, it is frequently necessary to acquire active variations and movements such as, for example, the heartbeat, and therewith dynamic events and processes.

At present, dynamic imaging normally is done using magnetic resonance tomography or ultrasound systems, since a continuous measurement or a repeated measurement at short intervals by means of an x-ray apparatus or a computed tomography apparatus would lead to an unacceptably high x-ray dose exposure of the patient due for the aforementioned reasons.

In this context it is known (for example for the guidance of biopsy needles) to implement a number of x-ray exposures by means of an x-ray apparatus or a computed tomography apparatus within short temporal intervals using an extremely low intensity of the x-ray radiation. This procedure is suitable only for the guidance of objects with an extremely high absorption such as, for example, objects made from metal. For a time-resolved analysis of subjects with low or medium x-ray absorption (such as, for example, human organs) this procedure is not suitable since in practice such subjects cannot be differentiated from the noise signal contained in the measurement signal given the use of extremely low intensity x-ray radiation.

### SUMMARY OF THE INVENTION

An object of the present invention is to provide a control device for a computed tomography apparatus as well as a method for controlling a computed tomography apparatus which, for the acquisition of volume datasets of the same subject within a short time span, achieve a significant reduction of the x-ray dose exposure for the examination subject. The acquisition of dynamic processes in subjects with low or medium absorption thus is made feasible. By this reduction of the x-ray dose exposure, the acquisition of movement cycles in the human body by using a computed tomography apparatus is enabled.

The above object is achieved in accordance with the invention by a control arrangement for a computed tomography apparatus, the control device being connected with an x-ray source of the computed tomography apparatus in order to regulate the intensity and/or dose of x-ray radiation emitted by the x-ray source, dependent on x-ray radiation received by the receiver unit, The control device contains, or has access to, a computer that acquires and stores volume datasets of an examination subject as a result of the x-ray radiation received by the receiver unit. According to the present invention, the computer acquires a number of volume datasets of the same subject in temporal succession in order to detect a temporal variation of the subject; and the computer automatically establishes a correlation between a stored earlier-obtained volume dataset of the same subject and the volume dataset to be directly achieved in real time, during the acquisition of this further volume dataset of the same subject using the x-ray radiation actively received by the receiver unit. The control device controls the x-ray source such that the x-ray radiation used for the acquisition of the volume dataset being currently acquired directly exhibits an intensity and/or dose that is inversely dependent on the established correlation.

The operation of the inventive control device thus is based on storing at least the preceding volume dataset is stored in the sequential acquisition of a number of volume datasets of the same subject within a short time span. During the acquisition of a subsequent new volume dataset, the x-ray radiation (and therewith the current measurement data) acquired by the receiver unit are continuously compared with the corresponding measurement data of the stored, preceding volume dataset in order to establish correlations.

In the case of a high correlation between the stored and the current acquired measurement data, the current x-ray radiation received by the receiver unit contains no new information relative to the stored, older volume dataset. In the case of a high correlation, the inventive control device therefore automatically controls the x-ray source such that the intensity or dose of the emitted x-ray radiation is made low.

If the correlation between the current x-ray radiation received by the receiver unit and the stored, older volume dataset is low, it is assumed that additional information is acquired. As a result, in the event of a low correlation the inventive control device automatically controls the x-ray source such that x-ray radiation is emitted with a high intensity or x-ray dose so that the newly-acquired volume data exhibit a sufficient quality.

Thus an increase or decrease of the intensity of the x-ray radiation emitted by the x-ray source by the inventive control device does not have to correspond to absolute values of the intensity or x-ray dose, but rather is related in a relative manner to a previously-calculated, optimal intensity or x-ray dose for the acquisition of a volume dataset of a specific subject. In the case of a low correlation, the x-ray source is automatically controlled by the inventive control device such that x-ray radiation is emitted with the optimal intensity or x-ray dose. In the case of a high correlation, the x-ray source is automatically controlled by the inventive control device such that x-ray radiation is emitted with an intensity or x-ray dose that is decreased relative to the optimal intensity or x-ray dose.

Since the intensity or the x-ray dose of the x-ray radiation used for the acquisition of a volume dataset is thus regulated by the inventive control device dependent on the aforementioned information acquisition, a significant reduction can be achieved of the total x-ray dose to which the subject is exposed.

The inventive control device thus allows the use of a computed tomography apparatus for the acquisition of a number of volume datasets from the same subject within a short time span, and thus for acquisition of a time-resolved sequence of a subject.

Preferably each volume dataset is composed of a number of projections (projection datasets), whereby the individual projection datasets contain the x-ray radiation received by the receiver unit of the computed tomography apparatus for different angle positions of the x-ray source and of the receiver unit relative to an examination region of the computed tomography apparatus, and thus relative to the subject. The computer in or accessed by the control device establishes the correlation between a stored volume dataset and the volume dataset to be directly acquired, by comparison of stored projection data with current projection data for the same subject and the same angle position of the x-ray source and the receiver unit relative to the examination region of the computed tomography apparatus, and thus relative to the subject.

Since the computer in the inventive control arrangement compares stored projection data of a preceding, older volume dataset with currently-acquired projection data of a volume dataset to be directly acquired for the same subject and the same angle setting of x-ray source and receiver unit relative to the subject, it is ensured that the information contained in the projection data with regard to the subject exhibits the same spatial perspective and position. Any differences thus can only arise due to a temporal change of the subject between the preceding, sorted volume dataset and the current volume dataset to be acquired. The computer of inventive control arrangement can determine this correlation in a particularly simple manner by a difference formation between the stored and current projection data for the identical angle position. As a result, the inventive control device controls the intensity of the x-ray radiation to be emitted by the x-ray source for the temporally successive projection data in a manner inversely dependent on the correlation of preceding stored projection data with the currently-acquired projection data for the respective identical angle position.

In a preferred embodiment, the control device or the computer also can be connected with a rotation device for the x-ray source and the receiver unit of the computed tomography apparatus in order to determine the current angle position of the x-ray source and receiver unit relative to the examination region of the computed tomography apparatus, and thus relative to the subject.

By connecting the control device with the rotation device of the computed tomography apparatus, the inventive control device or the computer can directly establish the current angle position of the x-ray source and the receiver unit relative to the examination region of the computed tomography apparatus. In this case, it is not necessary to derive the angle position indirectly (for example from the projection data or the volume dataset).

The control device or the computer preferably is also designed to detect the current angle position of x-ray source and receiver unit relative to the examination region of the computed tomography apparatus (and therewith relative to the subject and/or a current tube current of the x-ray source and/or the current intensity and/or x-ray dose of the x-ray radiation emitted by the x-ray source) and to store this current angle position together with a respective volume dataset.

By the detection of these parameters, the computer of the inventive control arrangement makes the currently-acquired volume dataset well-suited for establishing correlations with both preceding and future subsequent volume datasets. Furthermore it is ensured that the information required for a reconstruction of image data from each volume dataset are available.

In a preferred embodiment, the control device controls the x-ray source such that the intensity and/or dose of the x-ray radiation that are used for the acquisition of subsequent projection data of a volume dataset is/are inversely dependent on the established correlation of the preceding projection data of the same volume dataset with the corresponding projection data of the stored volume dataset.

The current projection data thus are compared with the projection data of a preceding volume dataset, of a preceding revolution of x-ray source and receiver unit around the subject for identical angle positions. The more significantly that the projection data differ (which, for example, can be established by the difference of the logarithms), the higher the intensity or x-ray dose for the x-ray radiation of the x-ray source that is set by the inventive control device to acquire the next projection data. Conversely, the intensity or x-ray dose of the x-ray radiation of the x-ray source set by the inventive control device is reduced when the correlation of the projection data is high. This inventive solution is based on the fact that, given projection data with high correlation (and thus given projection data that concern regions of the subject that have remained temporally constant), already-stored, preceding projection data can be used for reconstruction of an image of this region. No entirely new, high-resolution image must be acquired.

Furthermore, it is advantageous for the computer to automatically store the established correlation between a stored volume dataset of the same subject and the volume dataset to be directly acquired as a correlation factor in the current volume dataset to be acquired.

This correlation factor can be specified, for example, in the form of a percentage of correlation and, for example, can result from the difference of the logarithms of the projection data to be compared or directly from the difference of the projection data. Because the correlation is stored in the respective volume datasets in the form of a correlation factor for the respective projection data, using the correlation factor it can be established which projection data should be used for the image reconstruction from a preceding volume dataset, and which projection data contain temporal variations of the subject, and thus must be reconsidered in the image reconstruction.

In this case, the control device is preferably fashioned in order to use the correlation factor for reconstruction of a volume dataset.

This reconstruction can ensue, for example, by incorporation of an image generated using an older volume dataset into the image generated using the current volume dataset, the incorporation for various regions of the image respectively ensuing dependent on the correlation factor. If the correlation is 100% in a region of the image, the image generated using the older volume dataset can be completely adopted (used) in this region. By contrast, if the correlation is 0% in a region, only the projection data contained in the current volume dataset are used for the generation of the image. It is thereby evident that the incorporation can ensue non-linearly; for example, it can ensue logarithmically.

The above object also is achieved in accordance with the invention by a computed tomography apparatus having an x-ray source to emit x-ray radiation, whereby the intensity and/or dose of the x-ray radiation being adjustable; a receiver unit to receive the x-ray radiation emitted by the x-ray source, with an examination region for a subject being disposed between the x-ray source and the receiver unit; and having a control arrangement as described above.

According to the invention, such a computed tomography apparatus allows the acquisition of time-resolved sequences of a subject while keeping the x-ray exposure for the subject low.

The computed tomography apparatus has a rotation device to rotate the x-ray source and the receiver unit relative to the examination region of the computed tomography apparatus, whereby a volume dataset of a subject in the examination region is acquired by the receiver unit as a result of x-ray radiation received within one rotation of x-ray source and receiver unit around 360°.

The computed tomography apparatus preferably has a dose monitor, arranged between the x-ray source and the reception device, for determination of an x-ray dose emitted by the x-ray source. The dose monitor can be connected to the control device or the computer.

Such dose monitors are provided in nearly all conventional computed tomography apparatuses. Like the intensity, the x-ray dose determined by means of the dose monitor is well suited for determination and influencing of the x-ray exposure of the subject in an examination. In the adjustment of the x-ray dose of the x-ray radiation emitted by the x-ray source relative to the adjustment of the intensity of the x-ray radiation emitted by the x-ray source, it is advantageous that the temporal exposure of the subject by the x-ray radiation is also directly taken into account in the adjustment of the x-ray dose.

The above object also is achieved in accordance with the invention by a method for control of a computed tomography apparatus for the acquisition of a current volume dataset of an examination subject for whom a past volume dataset is stored, wherein each volume dataset is composed of a number of projection datasets, and the projection data represent the x-ray radiation received by a receiver unit of the computed tomography apparatus for different angle positions of an x-ray source of the computed tomography apparatus and the receiver unit relative to the examination region of the computed tomography apparatus, and thus relative to the subject. The method includes the following steps of controlling the x-ray source and the reception device of the computed tomography apparatus for the acquisition of current projection data for the current volume dataset to be acquired, automatically establishing of a correlation between the current projection data to be acquired and corresponding stored projection data of the earlier volume dataset of the same subject and the same angle position, and automatically controlling the intensity and/or dose of the x-ray radiation emitted by the x-ray source for acquisition of subsequent current projection data, such that the x-ray radiation used for the acquisition of the subsequent projection data to be acquired exhibits an intensity and/or dose that is inversely dependent on the established correlation of the current acquired projection data.

The step of controlling the x-ray source and the receiver device of the computed tomography apparatus for acquisition of current projection data preferably includes acquisition of a current angle position of x-ray source and receiver unit relative to the examination region of the computed tomography apparatus, and thus relative to the subject, and/or a current tube current of the x-ray source, and/or a current intensity and/or x-ray dose of the x-ray radiation emitted by the x-ray source, as well as storage of the acquired values together with the respective projection data.

In a preferred embodiment, the step of automatically establishing a correlation between the current acquired projection data and corresponding stored projection data includes storage of the established correlation as a correlation factor regarding the respective projection data in the volume dataset to be directly acquired.

In this case, it is advantageous for the method to furthermore include the step of reconstructing the respective current volume dataset using the stored correlation factor and the stored past volume dataset.

### DESCRIPTION OF THE DRAWINGS

FIG. 1 schematically illustrates a computed tomography apparatus in which the inventive control device is integrated.

FIG. 2 is a flowchart of a preferred embodiment of the inventive method.

### DESCRIPTION OF THE PREFERRED EMBODIMENTS

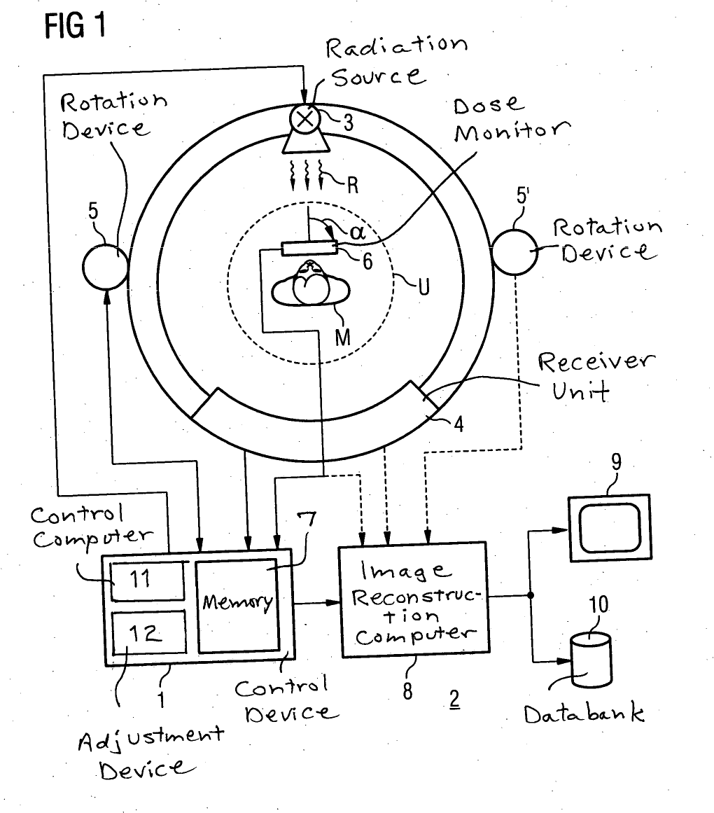

FIG. 1 schematically shows the design of a computed tomography apparatus 2 in which the inventive control device 1 is integrated. The control device in a first embodiment has a computer 11 and an adjustment unit 12.

The computer 11 of the control device 1 is connected with an x-ray source 3, a receiver unit 4, a rotation device 5 and a dose monitor 6 of the computed tomography apparatus 2.

Controlled by the adjustment unit 12 of the control device 1, the x-ray source 3 emits x-ray radiation R with a predetermined intensity and/or dose. The predetermined values for intensity and/or dose are not constant, but rather follow a predetermined time curve.

The x-ray radiation R emitted by the x-ray source 3 exposes an examination subject M (in an examination region U of the computed tomography apparatus 2) and the dose monitor 6. The x-ray radiation R, attenuated dependent on the material distribution and composition of the subject M, is subsequently detected by the receiver unit 4, which here is a semiconductor sensor. Electrical signals corresponding to the detected x-ray radiation are output by the receiver unit 4 to the control device 1 in the form of projection data.

The dose monitor 6 simultaneously outputs information about the emitted x-ray dose to the computer of the control device 1.

By means of the rotation device 5, the inventive control device 1 causes the x-ray source 3 together with the receiver unit 4 to rotate through 360° relative to the subject M in the examination region U.

During a rotation of the x-ray source 3 and the receiver unit 4 by approximately 360° relative to the subject M in the examination region U, the control device 1 acquires a volume data set for the subject M from the projection data output by the receiver unit 4.

The rotation device 5 is preferably a stepper motor, such that the rotation device 1 has precise knowledge in real time about an angle position α of the x-ray source 3 and the receiver unit 4 relative to the subject M. Alternatively, the control device can be designed (configured) in order to derive the angle position from the received projection data.

To acquire a time-resolved sequence of the subject M, the control device 1 controls the x-ray source 3 and the receiver unit 4 as well as the rotation device 5 such that a number of temporally subsequent volume data sets are acquired in a number of temporally subsequent revolutions of x-ray source 3 and receiver unit 4 around the subject M. The acquired volume data sets are stored in a FIFO memory 7 of the control device 1.

According to the present invention, during the acquisition of a volume data set of the same subject M using the x-ray radiation R currently received by the receiver unit 4, the computer 11 of the control device 1 automatically establishes correlations in real time between the preceding volume data set (stored in the FIFO memory 7) of the same subject and the volume data set to be directly acquired.

In the present example, this ensues by the computer 11 of the control device 1 comparing the projection data (contained in the volume data set stored in the FIFO memory 7) for the same subject M and the same angle position α of the x-ray source 3 and the receiver unit 4 with the projection data directly acquired by the receiver unit 4. In the present example, the projection data of the same spatial perspective and position of the same subject are compared. This ensues in the present example in that the difference of the logarithms is determined for the respective position data. Alternatively, it would be possible to determine the correlation by direct difference formation of the respective projection data.

For the acquisition of the subsequent projection data, the inventive control device 1 automatically controls the x-ray source 3, via the adjustment unit 12, such that the intensity and/or dose of the x-ray radiation emitted by the x-ray source 3 that is used for the acquisition of the subsequent projection data is inversely dependent on the established correlation. The more significantly that the projection data differ, the higher intensity or the higher the dose of x-ray radiation that is caused by the control device 1 to be emitted by the x-ray source 3 for the acquisition of the next projection data. Conversely, the control device 1 controls the x-ray source 3 automatically such that x-ray radiation with lower intensity or dose is requested for the acquisition of the next projection data when the correlation of the projection data is high.

In this context, the subsequent/next projection data can be either the projection data of the same volume data set that are adjacent (and thus immediately successive) with regard to the angle position α of the x-ray source and the receiver unit 4 relative to the subject M, or can be projection data for the identical angle position of a subsequent volume data set.

The control device 1 in the preferred embodiment shown in FIG. 1 thus controls the x-ray source 3, such that the intensity or dose of the x-ray radiation used for the acquisition of subsequent projection data of a volume data set is inversely dependent on the established correlation of the preceding projection data of the same current volume data set to be acquired with the corresponding projection data of the preceding volume data set stored in the FIFO memory 7.

In the preferred exemplary embodiment, during the acquisition of a volume data set the control device 1 automatically detects (via the rotation device 5) the current angle position α of the x-ray source 3 and the receiver unit 4 relative to the examination region U of the computed tomography apparatus 2 and, stores the angles with the projection data in the respective volume data set. Furthermore, in the present example the computer 11 of the control device 1 detects (by means of the dose monitor 6) the intensity and x-ray dose of the x-ray radiation emitted by the x-ray source 3 during the acquisition of a volume data set, as well as the tube current used for operation of the x-ray source 3, and also stores these values together with the associated projection data in the respective volume data set.

Furthermore, the control device 1 automatically stores the established correlation between the projection data contained in the preceding volume data set stored in the FIFO memory 7 and the directly-acquired projection data as a correlation factor in the current volume data set to be acquired. In the present example, the correlation factor is a dimensionless number that specifies the correlation as a percentage. Naturally, the correlation factor can be selected differently.

Furthermore, the computed tomography apparatus 2 shown in FIG. 1 has a reconstruction computer 8 for reconstruction of images.

For this purpose, the reconstruction computer 8 reads out the volume data sets stored in the FIFO memory 7 of the control device 1 and uses the correlation factor contained in the volume data sets for reconstruction of images from the volume data sets. In the present example, this ensues by means of a weighted averaging of the projection data that are contained in the volume data set forming the basis of a respective image and in the volume data set temporally preceding this volume data set. The weighting of the averaging ensues by means of the correlation factor. This has the result that the projection data contained in the preceding volume data set are incorporated with a high weighting in the reconstruction of the image in regions in which a high correlation exists between a current image and the preceding image. In regions in which only a slight correlation exists, the projection data contained in the preceding volume data set are incorporated into the new image to be generated with only a small weighting. Alternatively, a threshold for the correlation can be used. If the correlation lies below the threshold, the image is exclusively generated using the current projection data, but if the correlation lies above the threshold, the image is generated exclusively using the preceding projection data.

In FIG. 1, the reconstruction computer 8 is furthermore connected with a monitor 9 for output of the reconstructed images and a databank 10 for storage of the reconstructed images.

In an alternative embodiment that is shown with dashed lines in FIG. 1, the reconstruction computer 8 can also itself be connected with the receiver unit 4, and with a rotation sensor 5′ for the establishment of the angle position of the x-ray source 3 and receiver unit 4 relative to the subject M, and with the dose monitor 6.

In this case, the control device 1 is responsible only for the regulation of the intensity or dose of the x-ray radiation emitted by the x-ray source 3 and merely forwards the correlation factors belonging to the respective volume data sets to the reconstruction computer 8, which performs the aforementioned functions of the computer 11.

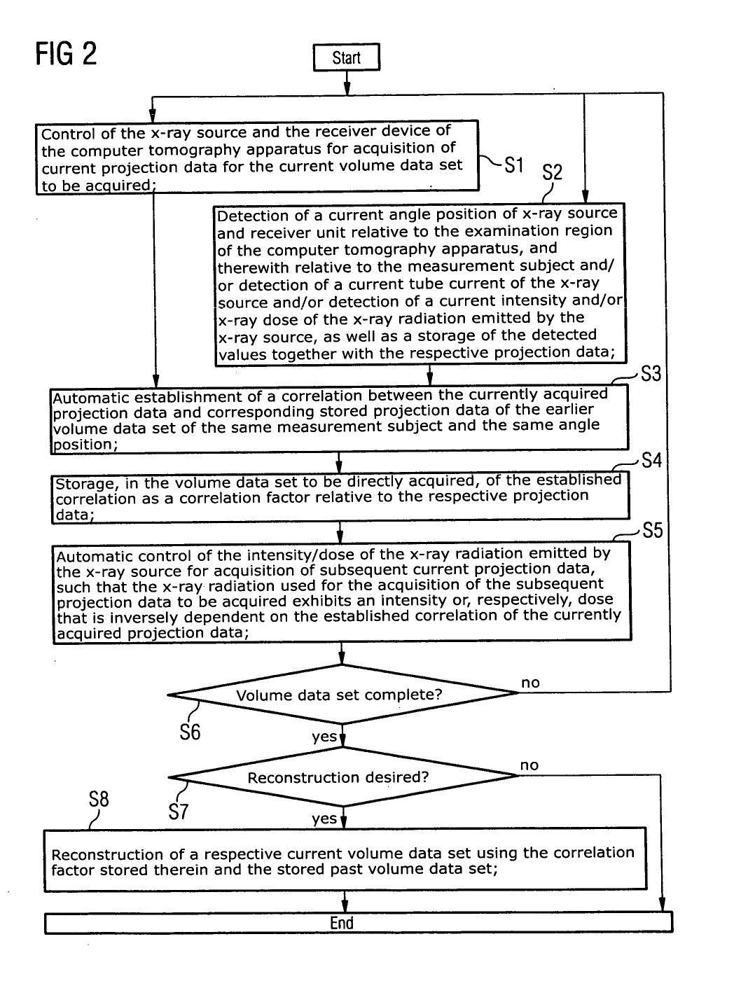

In the following, a preferred embodiment of the inventive method for controlling a computed tomography apparatus is described with reference to FIG. 2.

In a first step S2, the x-ray source and the receiver device of the computed tomography apparatus are controlled to acquire current projection data for a current volume data set to be acquired.

An acquisition of a current angle position of the x-ray source and the receiver unit relative to the examination region of the computed tomography apparatus (and therewith relative to the subject) and an acquisition of a current tube current of the x-ray source and a current intensity as well as x-ray dose of the x-ray radiation emitted by the x-ray source, and a storage of the acquired values together with the respective projection data, simultaneously ensue in step S2.

In a subsequent step S3, a correlation is automatically established between the current acquired projection data and corresponding stored projection data of an earlier volume data set of the same subject and the same angle position.

This correlation is stored in step S4 as a correlation factor regarding the respective projection data in the volume data set to be directly acquired.

The intensity and/or dose of the x-ray radiation emitted by the x-ray source for acquisition of subsequent current projection data is subsequently controlled such that the x-ray radiation used for the acquisition of the subsequent projection data to be acquired exhibits an intensity or, respectively, dose that is inversely dependent on the established correlation of the currently acquired projection data.

In step S6 it is subsequently determined whether the current volume data set to be acquired is complete. If this is not the case, the method returns back to the steps S1 and S2.

Otherwise, in the following step S7 it is determined whether a reconstruction of image data using the directly-acquired volume data set is desired. If this is not the case, the method terminates.

Otherwise, a reconstruction of the current volume data set ensues in step S8 using the correlation factor stored therein and the stored past volume data set in order to reconstruct an image before the method terminates.

The time sequence of the volume data sets to be acquired in succession by means of the method described in the preceding, which method is implemented by means of the computed tomography apparatus, is less than an hour and advantageously less than 10 minutes, and preferably less than 60 seconds.

In this context it should be emphasized that the steps S2 through S6 described in the preceding do not have to be sequentially, temporally separated from one another, but can also run in real time. In this case, sequential, successive, separate projection data of a volume data set are not acquired, but rather the projection data of the volume data set are continuously acquired. A connection of the data contained in the respective volume data set with the respective projection data is then produced via the respective angle position of the x-ray source and receiver unit during the acquisition of the respective projection data.

Since the inventive control device and the inventive method for control of a computed tomography apparatus upon implementation of temporally subsequent measurements for acquisition of volume data sets ensure that x-ray radiation with high dose or intensity is used only for acquisition of new information as a result of a temporal variation of a subject, the overall dose for the acquisition of successive volume data set can be kept as low as possible.

The use of a computed tomography apparatus for time-resolved acquisition of a subject thus is enabled. Furthermore, a savings of the load acting on the tube of the x-ray source is achieved, so the lifespan of the tube can be increased and the operating costs can be lowered. Furthermore, heating of the x-ray source in the immediately successive measurements can be kept low, so heating problems are avoided.

Although modifications and changes may be suggested by those skilled in the art, it is the intention of the inventor to embody within the patent warranted hereon all changes and modifications as reasonably and properly come within the scope of his contribution to the art.

I claim as my invention:

1. A control arrangement for a computed tomography apparatus, said computed tomography apparatus having an x-ray source that emits x-ray radiation, and having an examination region adapted to receive an examination subject for irradiation by said x-ray radiation in said computed tomography apparatus, said computed tomography apparatus also having a receiver unit that detects said x-ray radiation attenuated by the examination subject, said control device comprising:
a control computer in communication with said receiver unit to acquire and store a number of temporally successive volume datasets of said examination subject produced by said irradiation of said examination subject with said x-ray radiation and detecting a temporal change in said examination subject from said successive volume datasets, said control computer, during acquisition of a current volume dataset of said examination subject, automatically establishing a correlation, dependent on said temporal change, in real time between x-ray radiation currently being received by said receiver unit for said current volume dataset and a stored, earlier-acquired volume dataset of said examination subject; and
an adjustment unit connected to said x-ray source and operated by said control computer to adjust a characteristic of said x-ray radiation, selected from the group consisting of radiation intensity and radiation dose that is inversely dependent on said correlation.

2. A control arrangement as claimed in claim 1 wherein said computed tomography apparatus comprises a rotation device on which said x-ray source and said receiver unit are mounted, said rotation device rotating said x-ray source and said receiver unit around said examination region to irradiate the examination subject from a number of different angle positions relative to said examination region thereby producing, at each angle position, a projection dataset, and wherein said volume dataset is comprised of a number of said projection datasets, and wherein said control computer calculates said correlation for a current projection dataset being acquired in said current volume dataset and a projection dataset in said stored, earlier-acquired volume dataset that was acquired at the same angle as said currently acquired projection dataset.

3. A control arrangement as claimed in claim 2 wherein said control computer determines a current angle position for said projection dataset being currently acquired by analysis of said volume dataset being currently acquired.

4. A control arrangement as claimed in claim 2 wherein said control computer is connected to said rotation device and determines a current angle position of the projection dataset being currently acquired directly from said rotation device.

5. A control arrangement as claimed in claim 2 wherein said control computer stores, for each projection dataset, the respective angle position at which the projection dataset was acquired, in each stored volume dataset.

6. A control arrangement as claimed in claim 5 wherein said x-ray tube is operated with a tube current, and wherein said control computer, for each projection dataset in each volume dataset, stores at least one of said tube current, said radiation intensity, or said radiation dose, in each stored volume dataset.

7. A control arrangement as claimed in claim 1 wherein said control computer establishes said correlation using a volume dataset, as said stored, earlier-acquired volume dataset, that was acquired immediately preceding said current volume dataset.

8. A control arrangement as claimed in claim 1 wherein said control computer determines said correlation as a numerical correlation factor.

9. A control arrangement as claimed in claim 8 wherein said computed tomography apparatus comprises an image reconstruction computer that reconstructs an image of said examination subject from at least one of said volume datasets according an image reconstruction algorithm, and wherein said image reconstruction computer uses said correlation factor in said image reconstruction algorithm.

10. A control arrangement as claimed in claim 9 wherein said image reconstruction computer comprises said control computer.

11. A computed tomography apparatus comprising:
an x-ray source that emits x-ray radiation into an examination region adapted to receive an examination subject for irradiation by said x-ray radiation;
a receiver unit that detects said x-ray radiation attenuated by the examination subject;
a control computer in communication with said receiver unit to acquire and store a number of temporally successive volume datasets of said examination subject produced by said irradiation of said examination subject with said x-ray radiation and detecting a temporal change in said examination subject from said successive volume datasets, said control computer, during acquisition of a current volume dataset of said examination subject, automatically establishing a correlation, dependent on said temporal change, in real time between x-ray radiation currently being received by said receiver unit for said current volume dataset and a stored, earlier-acquired volume dataset of said examination subject; and
an adjustment unit connected to said x-ray source and operated by said control computer to adjust a characteristic of said x-ray radiation, selected from the group consisting of radiation intensity and radiation dose that is inversely dependent on said correlation.

12. A computed tomography apparatus as claimed in claim 11 comprising a rotation device on which said x-ray source and said receiver unit are mounted, said rotation device rotating said x-ray source and said receiver unit around said examination region to irradiate the examination subject from a number of different angle positions relative to said examination region thereby producing, at each angle position, a projection dataset, and wherein said volume dataset is comprised of a number of said projection datasets, and wherein said control computer calculates said correlation for a current projection dataset being acquired in said current volume dataset and a projection dataset in said stored, earlier-acquired volume dataset that was acquired at the same angle as said currently acquired projection dataset.

13. A computed tomography apparatus as claimed in claim 12 wherein said control computer determines a current angle position for said projection dataset being currently acquired by analysis of said volume dataset being currently acquired.

14. A computed tomography apparatus as claimed in claim 12 wherein said control computer is connected to said rotation device and determines a current angle position of the projection dataset being currently acquired directly from said rotation device.

15. A computed tomography apparatus as claimed in claim 12 wherein said control computer stores, for each projection dataset, the respective angle position at which the projection dataset was acquired, in each stored volume dataset.

16. A computed tomography apparatus as claimed in claim 15 wherein said x-ray tube is operated with a tube current, and wherein said control computer, for each projection dataset in each volume dataset, stores at least one of said tube current, said radiation intensity, or said radiation dose, in each stored volume dataset.

17. A computed tomography apparatus as claimed in claim 11 wherein said control computer establishes said correlation using a volume dataset, as said stored, earlier-acquired volume dataset, that was acquired immediately preceding said current volume dataset.

18. A computed tomography apparatus as claimed in claim 11 wherein said control computer determines said correlation as a numerical correlation factor.

19. A computed tomography apparatus as claimed in claim 18 comprising an image reconstruction computer that reconstructs an image of said examination subject from at least one of said volume datasets according an image reconstruction algorithm, and wherein said image reconstruction computer uses said correlation factor in said image reconstruction algorithm.

20. A computed tomography apparatus as claimed in claim 19 wherein said image reconstruction computer comprises said control computer.

21. A method for controlling a computed tomography apparatus, said computed tomography apparatus having an x-ray source that emits x-ray radiation, and having an examination region adapted to receive an examination subject for irradiation by said x-ray radiation in said computed tomography apparatus, said computed tomography apparatus also having a receiver unit that detects said x-ray radiation attenuated by the examination subject, said method comprising:
in a control computer in communication with said receiver unit acquiring and store a number of temporally successive volume datasets of said examination subject produced by said irradiation of said examination subject with said x-ray radiation and detecting a temporal change in said examination subject from said successive volume datasets, and in said control computer, during acquisition of a current volume dataset of said examination subject, automatically establishing a correlation, dependent on said temporal change, in real time between x-ray radiation currently being received by said receiver unit for said current volume dataset and a stored, earlier-acquired volume dataset of said examination subject; and
operating an adjustment unit connected to said x-ray source and said control computer to adjust a characteristic of said x-ray radiation, selected from the group consisting of radiation intensity and radiation dose that is inversely dependent on said correlation.

22. A method as claimed in claim 21 wherein said computed tomography apparatus comprises a rotation device on which said x-ray source and said receiver unit are mounted, said method comprising, with said rotation device, rotating said x-ray source and said receiver unit around said examination region to irradiate the examination subject from a number of different angle positions relative to said examination region thereby producing, at each angle position, a projection dataset, and wherein said volume dataset is comprised of a number of said projection datasets, and comprising, in said control computer, calculating said correlation for a current projection dataset being acquired in said current volume dataset and a projection dataset in said stored, earlier-acquired volume dataset that was acquired at the same angle as said currently acquired projection dataset.

23. A method as claimed in claim 22 comprising said control computer, determining a current angle position for said projection dataset being currently acquired by analysis of said volume dataset being currently acquired.

24. A method as claimed in claim 22 wherein said control computer is connected to said rotation device and comprising acquiring, in said control computer, a current angle position of the projection dataset being currently acquired directly from said rotation device.

25. A method as claimed in claim 22 comprising storing in a memory accessible by said control computer, for each projection dataset, the respective angle position at which the projection dataset was acquired, in each stored volume dataset.

26. A method as claimed in claim 25 comprising operating said x-ray tube with a tube current, and comprising storing, in said memory, for each projection dataset in each volume dataset, at least one of said tube current, said radiation intensity, or said radiation dose, in each stored volume dataset.

27. A method as claimed in claim 21 comprising, in said control computer, establishing said correlation using a volume dataset, as said stored, earlier-acquired volume dataset, that was acquired immediately preceding said current volume dataset.

28. A method as claimed in claim 21 comprising, in said control computer determining said correlation as a numerical correlation factor.

29. A method as claimed in claim 28 wherein said computed tomography apparatus comprises an image reconstruction computer and said method comprising, in said reconstruction computer, reconstructing an image of said examination subject from at least one of said volume datasets according an image reconstruction algorithm using said correlation factor in said image reconstruction algorithm.
